(19) United States
(12) Patent Application Publication
(10) Pub. No.: US 2011/0256499 A1
(43) Pub. Date: Oct. 20, 2011

(54) Dental Mirror

(76) Inventor: Randall Corey Snow, Pocatello, ID (US)

(21) Appl. No.: 12/762,578

(22) Filed: Apr. 19, 2010

(51) Int. Cl. A61B 1/247

(52) U.S. Cl. 433/30

(57) ABSTRACT

A dental mirror having an elongate handle and a mirror angularly disposed from one end of said handle. The mirror has an outer perimeter of at least two straight sides—which provides expanded surface area—which meet at a corner having a radius such that the corner retracts the cheek of a dental patient when the mirror is disposed in a viewing position in proximity to the outer side of the gums or teeth.

### BACKGROUND OF THE INVENTION

1. Field of the Invention

The present invention relates generally to dental mirrors and more specifically to an improved dental mirror that has a new improved configuration having the capability of retracting the tongue and cheek away from the teeth being examined or worked on by the dentist, all with comfort for and safety to the patient.

2. Description of Related Art

Conventional dental mirrors that are currently used by dentists today are round mirrors which are attached to a handle. The mirror has the advantage that it is relatively inexpensive to manufacture. However, it has certain disadvantages. The dental mirror is the most used dental instrument and is in a dentist's hand most of the time while the dentist is performing a dental procedure. About one half of the time that it is in use by a dentist it is used as a tissue retractor to retract a patient's tongue and/or cheek. In this regard the round mirror has some disadvantages. For example, when used as a tissue retractor the round edge of the prior art mirror has a very small surface area for retracting of the tongue which sometimes makes it difficult keeping the tongue away from the working area. Also, when retracting the cheek the mirror can slip from the cheek because of the round edge of the mirror.

An area that is particularly difficult to see and to work on is between a patient's rear molars and his/her cheek because of the relatively close spacing that exists between them. It is often quite uncomfortable for the patient and can result in injury to the mouth in the vicinity of the gums or cheek when the round mirror is used to push the cheek away from the gums in the region of the large molars.

A dental mirror that retracts a patients cheek is disclosed, for example, in
U.S. Pat. No. 4,859,180
Inventor: Smith, et al.
Issued: Aug. 22, 1989

This patent discloses dental mirrors that are self-drying and have the capability of retracting the cheek away from the teeth being examined or worked on by the dentist, all with comfort for and safety to the patient. The mirrors have clips or the like to detachably secure an absorbent roll thereon. The absorbent roll, such as a cotton roll commonly used in the practice of dentistry for absorbing fluids from various regions of the mouth, is preferably positioned and secured in a substantially tangential position relative to a circular or elliptically-shaped mirror. This tangential position in turn may be varied relative to the longitudinal axis of the elongated handle of the dental mirror, since the mirrors can be used for viewing and absorbing fluids from any region of the mouth. Such mirrors enable the dentist to retract the gum from the cheek by means of a soft absorbent roll rather than a hard surface such as the rim of a dental mirror which can and does cause discomfort and even injury to the patient. Concurrently the fluids in the vicinity of the absorbent roll are readily absorbed. Since the roll is in close proximity to the face of the mirror, this absorptive action in turn tends to keep the face of the mirror free or at least freer of fluids which would otherwise accumulate thereon. Consequently the ability of the dentist to view the area being examined or worked on is enhanced. Also, with properly designed clips it is a simple matter to replace wet rolls with fresh, dry rolls as needed during the examination or treatment being performed.

What is needed is a dental mirror which allows a dentist to overcome the problems of the mirror slipping when retracting a patients cheek and works as a better soft tissue retractor—like the tongue—because of increased surface area. Additional benefits also include decreasing the level of discomfort and injury when retracting a patient's cheek from the gums, both safely and comfortably for the patient.

### SUMMARY OF THE INVENTION

In an exemplary embodiment of the present invention, there is disclosed a dental mirror comprising:
an elongated handle; and
a mirror angularly disposed from one end of said handle;
wherein the mirror has an outer perimeter of at least two straight sides which meet at a corner having a radius such that the corner retracts a cheek of a dental patient when the mirror is disposed in a viewing position in proximity to the outer side of the gums or teeth.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a top view of a prior art round dental mirror;

FIG. 2 is a side view of the prior art dental mirror of FIG. 1;

FIG. 3 is a top exploded view of an embodiment of a new improved dental mirror in accordance with the principles of the invention;

FIG. 4 is a side exploded view of the embodiment of the dental mirror of FIG. 3 in accordance with the principles of the invention;

FIG. 5 is a top exploded view of another embodiment of a new improved dental mirror in accordance with the principles of the invention; and

FIG. 6 is a side exploded view of the embodiment of the dental mirror of FIG. 5 in accordance with the principles of the invention;

### DESCRIPTION OF THE PREFERRED EMBODIMENT

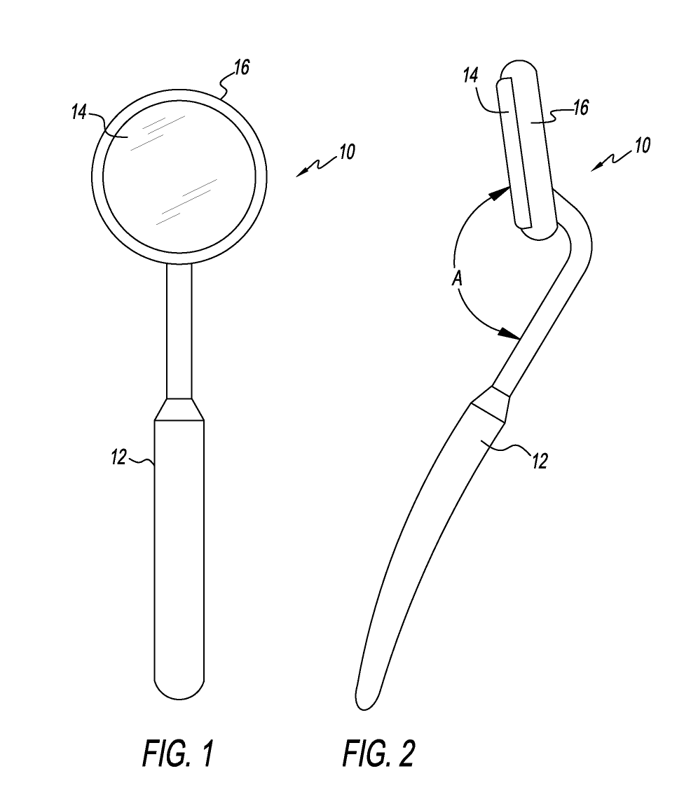

Referring to FIGS. 1 and 2, there is shown a top and side view of a prior art round dental mirror currently used by almost all dentists. The dental mirror 10 includes an elongate handle 12 which is attached at one end to a backing 16 for a mirror 14. The face of the mirror is oriented to reside at an angle “A” of approximately 135 degrees, more of less relative to the handle as is shown in FIG. 2. Mirror 14 and the backing 16 for the mirror are circular.

The face of the mirror is made of glass or a highly reflective metallic surface and the handle 12 can have a cross sectional shape that is hexagonal, round, square, or any other desired cross sectional shape.

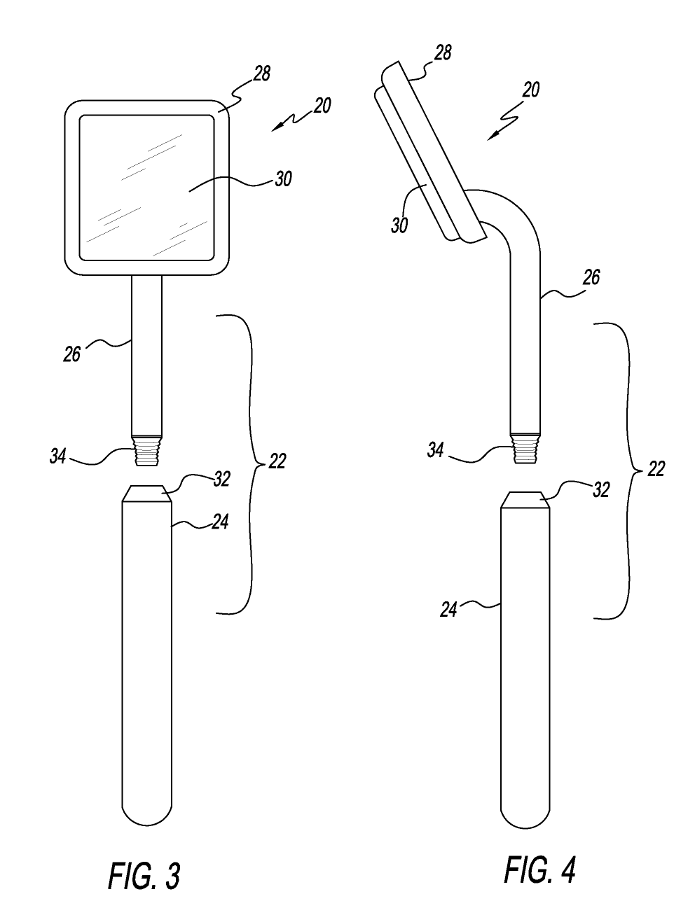

Referring to FIGS. 3 and 4, there is shown top and side views of a square dental mirror in accordance with the principles of the invention. The dental mirror is the instrument that a dentist uses most. It is used by a dentist while doing a procedure on a patient, and about half of the time that it is being used it is used as a tissue retractor to reposition or move a patient's tongue or cheek. In counter-distinction to a round mirror, the straight edge and corner of the new improved square dental mirror shown in FIGS. 3, 4 provides more surface area when the mirror is used to retract a patient's tongue and provides the dentist with a corner gripping surface that can be used to retract a patent's cheek The corners of the mirror are slightly rounded for a patient's comfort and the angle of the mirror with respect to the handle can be the same as currently manufactured prior art round dental mirrors.

Continuing with FIGS. 3 and 4, there is shown top and side expanded views of an embodiment of the invention where the dental mirror has a square shape. The dental mirror 20 includes an elongated handle 22 which can consist of a handle member 24 attached at one end to a screw-in mirror member 26. The face of the mirror is oriented to reside at an angle of approximately 135 degrees, more or less relative to the handle. The screw-in mirror member 26 includes a mirror backing 28 that is attached to a mirror 30. In this embodiment the mirror 30 and the backing 28 for the mirror have a shape that is square. It is understood that in another embodiment the shape of the mirror and mirror backing can be rectangular. The face of the mirror is made of glass or a highly reflective metallic surface and the handle member 24 can have a cross sectional shape that is hexagonal, round, square, or any other desired cross sectional shape.

The handle member 24 and the screw-in mirror member 26 can be a single continuous unit of any suitable material, such as stainless steel where the mirror 30 is made of glass that has a high reflecting coating on its rear surface.

In another embodiment the elongated handle 22 can be made of two separate members, a handle member 24 and a screw-in mirror member 26 where the screw-in mirror member is adapted to be threaded into the handle member to form a unitary member. In this embodiment handle member 24 includes a coupling end 32 such as a threaded receiving chamber located at an end of the handle member 24 adapted to receive a threaded end 34 of the screw-in mirror member 26. The screw-in mirror member comprises a single piece mirror backing 28 which is attached to a shaft having the threaded end 34 which is adapted to be threaded into the threaded receiving chamber at the coupling end 32 of the handle member 24.

The screw-in mirror member can be formed of a semi-rigid curable polymer material such as polyvinyl chloride so that the screw-in mirror member 26, the portion of the dental mirror that is placed into a patient's mouth can be disposed after use to eliminate sterilization problems. In addition, when not assembled the two part dental mirror is compact having an overall length of less than seven inches.

Continuing with FIGS. 3 and 4, screw in mirror member 26 includes a mirror backing 28 that has a flat recessed upper surface to which the rear surface of mirror 30 is mounted with a water proof adhesive such as an epoxy. The mirror backing is attached to a shaft which projects out from mirror backing at an angle of between thirty to sixty degrees, and preferably at an angle of about 45 degrees more of less. This angle can be the same angle that is used with prior art round mirrors which are currently being used. To use the dental mirror here disclosed a dentist needs only to thread the end of the screw in mirror member into the threaded chamber in the end of handle member 24. Following use on a patient, the screw-in mirror member can be removed from the handle member and discarded.

The mirror 30 of the screw-in mirror member is a square mirror having rounded corners. Each corner can have a radius of between one sixty fourth and one quarter of an inch where a radius of about one sixteenth of an inch is preferred to avoid causing any discomfort to a dental patient. Each side of the square mirror can be between 1.8 cm and 2.6 cm where a length of 2.2 cm more or less on each side of the square is preferred. In this embodiment the various parts of the dental mirror can be composed of the same materials as prior art dental mirrors, that being stainless steel and rhodium-layered glass. It is understood that the mirror can, in another embodiment, have a shape that is rectangular.

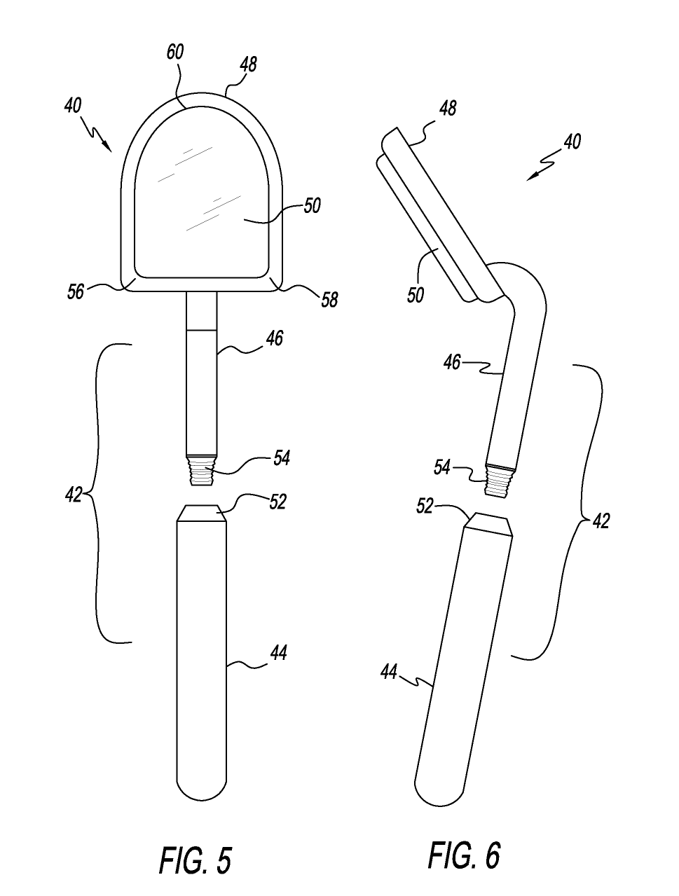

Referring to FIGS. 5 and 6, there is shown a top view of another embodiment of a dental mirror where the mirror is shaped like a spade type design wherein a traditional circular design has been elongated—resulting in a more straightened edge at the top and a rounded edge at the bottom end in accordance with the principles of the invention. The dental mirror is the instrument that a dentist uses most. It is used by a dentist most while doing a procedure on a patient, and about half of the time that it is being used by a dentist, it is being used as a tissue retractor to reposition or move a patients tongue or cheek. In counter-distinction to a round mirror, the straight edge and corner of the new improved spade shaped dental mirror shown in FIGS. 5, 6 provides more surface area when the mirror is used to retract a patient's tongue and now provides the dentist with a corner gripping surface that can be used to retract a person's cheek The corners of the mirror are rounded for a patient's comfort and the angle of the mirror with respect to the handle can be the same as currently manufactured prior art round dental mirrors.

Continuing with FIGS. 5 and 6, there is shown a top expanded view of an embodiment of the invention where the dental mirror has a spade shape. The dental mirror 40 includes an elongate handle 42 which can consist of a handle member 44 that is attached at one end to a screw-in mirror member 46. The face of the mirror is oriented to form an angle of approximately 135 degrees, more of less, relative to the handle. The screw-in mirror member 46 includes a mirror backing 48 that is attached to a mirror 50. In this embodiment mirror 50 and backing 48 for the mirror have a shape that is substantially that of a spade. The face of the mirror is made of glass or a highly reflective metallic surface and the handle member 44 can have a cross sectional shape that is hexagonal, round, square, or any other desired cross sectional shape.

The handle member 44 and the screw-in mirror member 46 can be a single continuous unit of any suitable material such as stainless steel where the mirror 50 is made of glass that has a high reflecting coating on its rear surface.

In another embodiment the elongated handle 42 can be made of two separate members, a handle member 44 and a screw-in mirror member 46 where the screw-in mirror member is adapted to be threaded to the handle member to form a unitary member. In this embodiment handle member 44 includes a coupling end 52 such as a threaded receiving chamber located at an end of the handle member 44 adapted to receive the threaded end of the screw in mirror member 46. The screw-in mirror member is a single piece mirror backing 48 attached to a shaft having a threaded end 54 adapted to be threaded into the threaded receiving chamber at the coupling end 52 of the handle member 44.

The screw-in mirror member can be formed of a semi-rigid curable polymer material such as polyvinyl chloride so that the screw-in mirror member 46, the portion of the dental mirror that is used in a patient's mouth can be disposed after use to eliminate sterilization problems. When not assembled, the two part dental mirror has an overall length of less than seven inches.

Continuing with FIGS. 5 and 6, screw in mirror member 46 includes a mirror backing 48 that has a flat recessed upper surface to which the rear surface of mirror 50 is mounted with a water proof adhesive such as an epoxy. The mirror backing is attached to a shaft which projects out from mirror backing at an angle of between thirty to sixty degrees, and preferably at an angle of about 45 degrees more of less. This angle can be the same angle that is used with prior art round mirrors which are currently being used. To use the dental mirror here disclosed a dentist need only thread the threaded end of the screw in mirror member into the threaded chamber at the coupling end of the handle member 44. Following use on a patient, the screw in mirror member can be removed from the handle member and discarded.

The mirror 50 of the screw in mirror member is spade shaped with three rounded corners 56, 58, 60. Corners 56, 58 can have a radius between one sixty fourth and one quarter of an inch where a radius of about one sixteenth of an inch more or less is preferred to avoid causing any discomfort to a dental patient. Corner 60 has a more generous radius that can be between one quarter of an inch and three quarters of an inch where a radius of one half of an inch is preferred. Each side edge of the spade shaped mirror can be between 1.8 cm and 2.6 cm where a length of 2.2 cm more or less of each side edge of the spade shape is preferred. In this embodiment the various parts of the dental mirror can be composed of the same materials as prior art dental mirrors, that being stainless steel and rhodium-layered glass.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A dental mirror comprising:
an elongate handle; and
a mirror angularly disposed from one end of said handle;
wherein said mirror has an outer perimeter of at least two straight sides which meet at a corner having a radius such that said corner retracts a cheek of a dental patient when said mirror is disposed in a viewing position in proximity to the outer side of a gum.

2. The dental mirror of claim 1 wherein said radius is between one sixty fourth of an inch and one quarter of an inch.

3. The dental mirror of claim 1 wherein said radius is between one thirty second on an inch and fifteen sixty fourth of an inch of an inch.

4. The dental mirror of claim 1 wherein said radius is between one thirty second on an inch and eight thirty seconds of an inch.

5. The dental mirror of claim 4 wherein said dental mirror is a square mirror having four sides of similar lengths.

6. The dental mirror of claim 5 wherein the length of each side is between 1.8 cm and 2.6 cm.

7. The dental mirror of claim 5 wherein the length of each side is between 2 cm and 2.4 cm.

8. The dental mirror of claim 5 wherein the length of each side is 2.2 cm.

9. The dental mirror of claim 4 wherein said dental mirror is shaped like a spade having three straight sides and a rounded fourth side all connected with rounded corners.

10. The dental mirror of claim 9 wherein a first side is disposed from one end of said handle, a second side extends at an acute angle from a first end of said first side forming a first corner having a first radius, a third side extends at an acute angel from a second end of said first side forming a second corner having a second radius, and ends of said second and third sides meet forming a third corner having a third radius.

11. The dental mirror of claim 10 wherein said radius of said first and second corners are equal.

12. The dental mirror of claim 11 wherein said radius of said third corner is between one quarter and three quarters of an inch.

13. The dental mirror of claim 12 wherein said radius of said first and second corners are between one sixty fourth of an inch and one quarter of an inch.

14. The dental mirror of claim 12 wherein said radius of said first and second corners are between one thirty second on an inch and fifteen sixty fourth of an inch of an inch.

15. The dental mirror of claim 12 wherein said radius of said first and second corners are between one thirty second on an inch and eight thirty seconds of an inch.

16. The dental mirror of claim 13 wherein the length of each side is between 1.8 cm and 2.6 cm.

17. The dental mirror of claim 13 wherein the length of each side is between 2 cm and 2.4 cm.

18. The dental mirror of claim 13 wherein the length of each side is 2.2 cm.

19. The dental mirror of claim 11 wherein said radius of said third corner is one half of an inch.
